(19) United States
(12) Patent Application Publication
(10) Pub. No.: US 2007/0098769 A1
(43) Pub. Date: May 3, 2007

(54) Systems and methods for treating hot flashes associated with menopause

(76) Inventor: Mary J. Champion, Seal Beach, CA (US)

Correspondence Address: Frank J Uxa, 4 Ventura, Suite 300, Irvine, CA, 92618, US

(21) Appl. No.: 10/576,803

(22) PCT Filed: Apr. 11, 2005
(86) PCT No.: PCT/US05/12274

§ 371 Date: Apr. 21, 2006

Related U.S. Application Data
(60) Provisional application No. 60/563,237, filed on Apr. 16, 2004.

(51) Int. Cl. A61F 13/02; A61N 1/39

(52) U.S. Cl. 424/448; 607/1

(57) ABSTRACT

Kits and systems for treating hot flashes in a menopausal woman include one or more cooling devices suitable to be placed on a woman's skin, at a hot flash origin site, such as a location on the woman's back, for example, between the shoulder blades of the woman when experiencing a hot flash or in anticipation of a hot flash. Instructions for so applying such cooling devices may also be included. The cooling device or devices may be provided in a sealed package. The cooling devices may be in the form of an adhesive patch that can adhere to the woman's skin. A method for treating hot flashes includes providing one or more cooling devices, and instructing a woman to place the cooling device or devices on her back between her shoulder blades, for example.

### RELATED APPLICATION

This application claims the benefit of U.S. Provisional Application No. 60/563,237 which is hereby incorporated in its entirety herein by reference.

### BACKGROUND

This invention relates generally to treating one or more symptoms associated with menopause in a woman, for example, a menopausal woman. In particular, the invention relates to kits, systems and methods for treating one or more symptoms of hot flashes in such a woman.

Typically, hot flashes associated with female menopause, which hot flashes can have substantial or even debilitating adverse effects on affected women, have been treated with hormones. However, hormone therapy may be associated with numerous serious side effects. Thus, hormone therapy is often not recommended for the treatment of hot flashes in menopausal women. Alternatives in treating such hot flashes are needed, for example, to reduce, control, and/or manage one or more adverse effects or symptoms of such hot flashes.

U.S. Pat. No. 4,900,554 discloses an adhesive device for application to body tissue to provide sustained drug release or to protect a body tissue, the device having an adhesive layer and a backing layer positioned over one side of the adhesive layer. The adhesive layer includes one or more acrylic acid polymers having adhesive properties upon dissolution or swelling in water, and at least one water insoluble cellulose derivative. The backing layer is water insoluble or sparingly water soluble.

U.S. Pat. No. 5,780,047 discloses a patch that comprises a water-soluble adhesive sheet, and a water-soluble protective material laminated thereon. The patch can be applied to the skin so as to exhibit warm-bathing effects on the application site.

U.S. Pat. Nos. 6,224,899; 6,228,376; and 6,524,612 disclose an adhesive cooling gel composition which contains a large amount of water and which provides a cooling effect and/or coolness-preserving effect, and a process for preparing the same. The composition comprises a polyacrylic acid compound, a polyvalent metal component and water, wherein the content of water is 75 to 95% by weight based on the cooling composition. Also disclosed are an adhesive cooling composition shaped into a sheet, and a process for preparing the adhesive cooling composition. The process is selected from processes disclosed in the patent. Each process includes a step of mixing and dissolving the components of the composition and a deaerating step. In one process an aqueous solution is deaerated and then the other components of the composition are mixed and/or dissolved. In another process an aqueous solution is deaerated, and the other components of the composition are mixed and/or dissolved. In an additional process, the other components of the composition are mixed and/or dissolved, and the obtained solution is deaerated.

U.S. Pat. No. 5,956,963 discloses a wrist cooler that offers relief for hot flash symptoms of menopause and body overheating. The cooler includes chemical cooling pellets that remain in a solid state until broken. A woman experiencing one or more symptoms of menopausal hot flashes carries the cooler in her purse. When a hot flash occurs, the cooler is removed from her purse, the pellets are broken and the cooler is slid onto her wrist. Once the flash has subsided, the cooler is removed from the wrist and is discarded. This wrist cooler is easily transportable and provides for immediate relief, as there are no reusable cooling elements to be frozen or chilled. However, although the wrist coolers provide cooling in the wrist area, such coolers have been found to have relatively little effectiveness in otherwise reducing the severity of hot flashes. In addition, since a woman's wrist is often in plain view of others, wearing such wrist coolers may indicate to others, to the detriment of the wearer, that the wearer is experiencing a hot flash.

U.S. Patent Publication No. 2003/0176904 discloses a self-adhering cotton fabric cold strip that is to be worn as a wrist band, or an ankle band. The self-adhering cotton fabric cold strip has an envelope defining a sealed cold strip volume. A cooling gelatinous material is positioned in the cold strip volume. A bandage sheet is fixed to the envelope. The bandage sheet defines a bandage adhesive for temporary adhesion of the cold strip to the skin surface of a user. The self-adhesive envelope contains a cooling gelatinous material which reduces the body temperature. The reduction of the body temperature brings relief that is convenient, portable, and lasts for hours. The cold strip is to be worn as a wrist or ankle band for a period of hours and is then disposed and replaced with another cold strip. The self-adhering cotton fabric cold strip will be produced with different colors of cloth.

In view of the disadvantages of hormone therapy, new, and advantageously enhanced or improved, treatments are needed to control and/or reduce hot flash symptoms in menopausal women.

### SUMMARY OF THE INVENTION

The present invention is generally directed to non-hormonal treatments of hot flashes. Kits, systems and methods are described which involve placing a cooling device on a woman, advantageously on an inconspicuous location, and preferably on a torso and more preferably on an upper portion of a back, of the body of a woman who is experiencing a hot flash or who is prone to experiencing a hot flash, for example, as a result of menopause.

Although the exemplary embodiment of the invention is directed to placing the cooling device on the back of a woman, the cooling device may be placed on any site of a woman's body, preferably on or at any hot flash origin site, or a site where the hot flash, for example, the first symptom of the hot flash, is felt first. Such origin sites can be any place on the body. For example, the origin site may be on the upper portion of a woman's body, for example, the upper portion of the woman's torso, the woman's arms, the woman's head, the woman's neck, and the like, or a lower portion of a woman's body, for example, the lower portion of the woman's torso, the woman's legs and the like. In certain situations or embodiments, the hot flash origin site is other than the woman's wrists, or ankles, or chest. In certain situations or embodiments, the origin site is a location on the front of a woman's torso, for example, on a woman's stomach, in proximity to the elbow or elbows of a woman's arm or arms, on a woman's neck, or on the back of a woman's torso, for example, on a woman's upper and/or lower back. The origin site may vary from hot flash to hot flash and/or from woman to woman.

Placing the cooling device at the origin site of the hot flash enhances the usefulness of the cooling device to treat or mitigate against the overall adverse effects or symptoms of the hot flash, for example, relative to placing the cooling device on a woman's wrist when the wrist is not the origin site of the hot flash. Also, if an individual woman, based on her individual experience with her hot flashes, is aware that such hot flashes, for example, the first symptoms of such hot flashes, are often first felt at a specific body location, she can be ready, at the first sign of a hot flash, to apply the cooling device to that location. Still further, she may apply the cooling device to that location, that is a frequent hot flash origin site, prior to the start or onset of hot flash symptoms to even further reduce the severity of the hot flash, should such hot flash occur.

In one aspect of the invention, kits for treating hot flashes, for example, hot flashes associated with menopause in a woman, comprise one or more cooling devices; and instructions for using the cooling device or devices. The instructions preferably include directions to place the cooling device on the skin of a woman at a hot flash origin site, such as a location on her back, for example, between her shoulder blades, when a hot flash begins and/or in anticipation of a hot flash occurring.

In an additional aspect of the present invention, systems for treating hot flashes associated with menopause in a woman are provided. Such systems comprise at least one cooling device, preferably a plurality of cooling devices, having a contact surface, preferably a non-rectangular contact surface. The cooling device or devices are effective, when the contact surface of the cooling device or devices are placed in contact with a woman experiencing a hot flash or prone to experiencing a hot flash at a hot flash origin site of the woman, to treat at least one symptom of the hot flash.

In another aspect of the invention, methods for treating hot flashes, for example, hot flashes associated with menopause in a woman, comprise providing at least one cooling device; and instructing a woman to place the at least one cooling device on her skin at a hot flash origin site, such as a location on her back, for example, between the shoulder blades, when the hot flash begins and/or in anticipation of a hot flash occurring.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional advantages and aspects of the present invention are apparent in the following detailed description, drawings, and claims.

### BRIEF DESCRIPTION OF THE DRAWINGS

FIG. 1 is a schematic illustration of a kit or system for treating hot flashes.

FIG. 2 is a top plan view of a cooling device provided in the kit or system of FIG. 1.

FIG. 3 is a bottom plan view of the cooling device of FIG. 2.

FIG. 4 is a side view along line 4-4 of FIG. 3.

FIG. 5 is an illustration of two cooling devices located on the back of a menopausal woman.

FIG. 6 is a top plan view of an additional embodiment of a cooling device in accordance with the present invention.

FIG. 7 is a bottom plan view of the cooling device of FIG. 6.

FIG. 8 is a top plan view of a further embodiment of a cooling device in accordance with the present invention.

FIG. 9 is a bottom plan view of the cooling device of FIG. 8.

FIG. 10 is a side view of the cooling device of FIG. 8.

### DETAILED DESCRIPTION

A kit for treating hot flashes, for example, hot flashes associated with menopause in a woman, comprises at least one cooling device; and instructions for using the cooling device to treat hot flashes, for example, to reduce and/or control and/or manage, the severity of at least one symptom of a hot flash. The instructions preferably include directions to place the at least one cooling device on the skin of a woman at a hot flash origin site when the woman experiences a hot flash, such as when a hot flash begins, or when the woman is anticipating the onset or beginning of a hot flash. A system for treating hot flashes in accordance with the present invention may be considered to comprise one or more cooling devices, as described elsewhere herein.

Although the exemplary embodiment of the invention is directed to placing the cooling device on the back of a woman, as set forth elsewhere herein, the cooling device may be placed on any region or area of the woman's body, for example, at any hot flash origin site, or the site where the hot flash is felt first. Advantageously, in one embodiment the cooling device is placed on a region or area of the woman's body that is at or above the ankles and wrists, for example, at or above the knees and elbows. In one embodiment, the cooling device is placed on the woman's torso.

In one embodiment, the cooling device is placed on an inconspicuous region or area of the woman's body, that is a region of the woman's body that can carry the cooling device without the cooling device being visually or otherwise apparent to others. In one embodiment, the region or area of the woman's body on which the cooling device is placed is covered or shielded by clothing and/or is other than the upper portion of the woman's chest.

Hot flash origin sites can be any place on the body, and may vary from woman to woman, and even from hot flash to hot flash in each individual woman. For example, the hot flash origin site or body location where the hot flash or first symptom of the hot flash is felt may be on the upper portion of a woman's body or a lower portion of a woman's body. In certain situations, the origin site is any location other than the wrists, ankles, or chest. In certain situations, the origin site is a location on a woman's stomach, in proximity to the elbow of the woman's arms, on the woman's neck, or on the woman's back.

Typically, the cooling device is placed on the woman's body, for example, on the hot flash origin site, as soon as possible after a hot flash begins, such as within about five minutes or less from when a hot flash begins, for example, within about two minutes or less from when a hot flash begins. In addition, in certain instances it may be possible for the woman to anticipate a hot flash occurring before the onset of the hot flash or hot flash symptoms. For example, a woman may be more prone to hot flashes at certain times of the day or month, and/or under certain conditions of stress, excitement, etc. Thus, a woman may be able to anticipate that a hot flash is likely to occur in the near future. In this circumstance, for example, the cooling device is placed on the woman's body, for example, on a site of the woman's body where symptoms of hot flashes frequently first become apparent (for the particular woman involved), that is a hot flash origin site, in anticipation of a hot flash occurring. Such preemptive cooling mitigates against the effects or symptoms of a hot flash, should one occur.

In one embodiment, for example, when the cooling devices are used in anticipation of a hot flash occurring, two or more cooling devices may be applied to different regions or areas of the woman's body, that is different hot flash origin sites, such as when the woman has previously experienced hot flashes which had different origin sites.

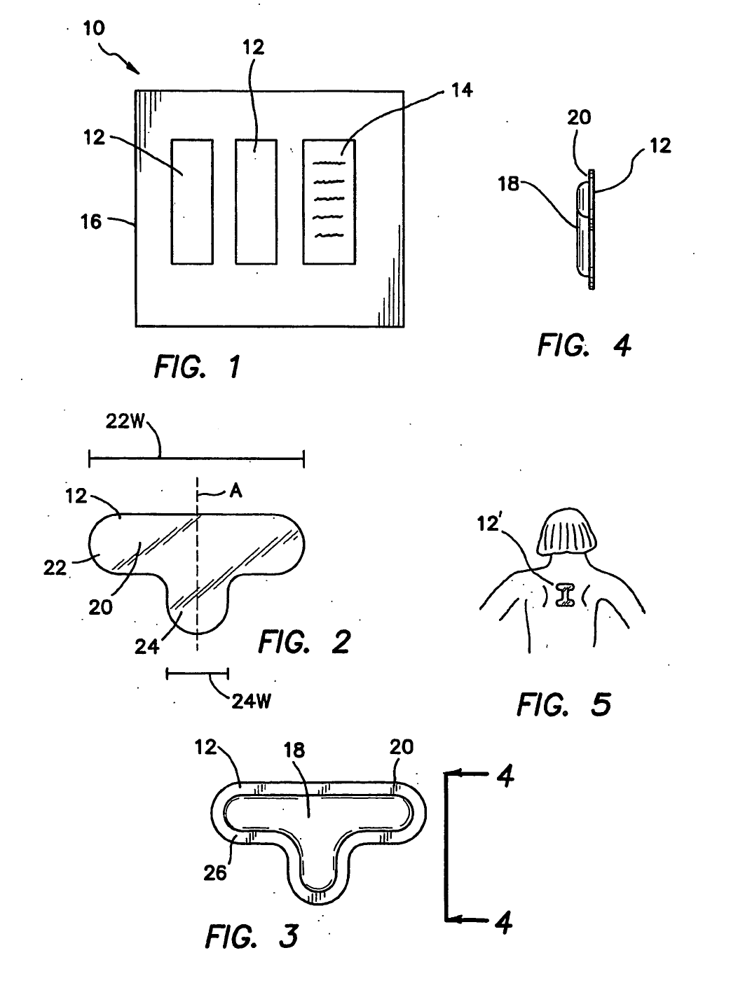

In reference to FIG. 1, a kit or system 10 in accordance with the present invention comprises a plurality of cooling devices 12. Instructions for use 14 of the cooling devices 12 are advantageously included in kit 10. The kit or system 10 is in the form of a sealed package 16. Although two cooling devices 12 are illustrated in the kit or system 10, the kit or system 10 may include only one cooling device 12, or more than two cooling devices 12. In addition, each cooling device 12 may be provided in an individual sealed package, for example, provided within the sealed package 16.

The cooling device 12 of the kit or system 10 may be any suitable device configured to cool the skin of a woman experiencing a hot flash. For example, many of the cooling devices described or identified elsewhere herein, for example, in the patents incorporated by reference herein, may be used in accordance with certain aspects of the present invention. The cooling device 12 is advantageously self-contained in that it provides cooling when applied to the skin of a living human without the addition of one or more components to the cooling device as provided, for example, as removed from a package. In one embodiment, the cooling device 12 may, be a device that can be placed in a freezer, refrigerator, and the like, and then removed therefrom, and placed on the skin at a cooled temperature (e.g., a temperature below room temperature, such as below about 70° F.). One example of such a cooling device is a so-called “ice-pack” or “cold-pack”. Alternately, the cooling device 12 may be a device configured to cool the woman's skin by removing heat from the woman's skin, such as by evaporation, endothermic chemical reaction, etc.

Any suitable cooling device may be used in accordance with the present invention. Such devices preferably provide for relatively rapid cooling when placed in contact with a woman's skin, for example, cooling within less than about 30 minutes or less than about 10 minutes or less than about 5 minutes of initial skin contact. Preferably, the amount of cooling should be sufficient to mitigate against at least one symptom of the hot flash, without causing undue or even significant discomfort and/or harm to the woman. For example, the device preferably does not cool the skin or body of the woman to a point at which the woman experiences significant discomfort because of the cooling. Moreover, the device itself should have no significant detrimental effect on the woman, for example, on the skin of the woman.

One example of a cooling device 12 is illustrated in FIG. 2. As illustrated, the cooling device 12 of FIG. 2 is symmetrical about an axis A. The cooling device 12 includes a first portion 22 and a second portion 24. The second portion 24 extends from the first portion 22. The first portion 22 has a width 22W, and the second portion 24 has a width 24W. The width 22W and the width 24W are substantially perpendicular to the axis A. The width 24W is illustrated as being smaller than the width 22W. The cooling device 12 is generally shaped as a T.

As shown in FIG. 3, the cooling device 12 comprises a cooling component 18 coupled to a substrate 20. The substrate 20 typically comprises a gas-permeable material, such as a natural or synthetic fiber material. In certain cooling devices, the substrate 20 comprises, consists essentially of, or consists of a fibrous sheet, for example, a cotton fiber sheet. The cooling component 18 is surrounded by a border 26 of the substrate 20. The border 26 is substantially free of the cooling component 18. In other embodiments, the cooling component 18 may extend to, or even outwardly beyond, at least a portion, or even all, of the outer periphery of substrate 20. Such embodiments advantageously provide increased cooling per unit area of the overall cooling device relative to a substantially identical device having a border free of cooling component. This feature may also allow the use of a smaller cooling device relative to a substantially identical device having a border free of cooling component. A smaller cooling device is advantageously less conspicuous when applied to a woman's skin.

In certain embodiments, the border 26 may include one or more adhesive regions effective in facilitating adhesion of the cooling device 12 on the woman's skin. The adhesive regions may include the same material of the cooling component 18, or may include other adhesive materials, such as topically acceptable glues, and the like. In other embodiments, the cooling component 18 may be sufficiently self-adhering so as to be effectively adhered to the woman's skin without the use of one or more separate adhesives. Thus, the border 26 may be substantially free of adhesive(s) or adhesive region(s).

FIG. 4 illustrates the thickness of the cooling device 12. The thickness of the cooling device 12 is determined by the thickness of the substrate 20 and the cooling component 18. Preferably, the thickness of the cooling device 12 is relatively small so that the cooling device 12 is not substantially visible, and preferably is not otherwise observable or conspicuous, through a woman's clothing when the device is placed on the woman's skin.

The cooling device 12 of the kit 10 may be an adhesive patch. The patch should be tacky or sticky enough to permit a woman to wear the cooling device 12 on her skin for extended periods of time, if desired. For example, the patch may adhere to the woman's skin for more than one minute, and may adhere to the woman's skin for about six hours or more. In addition, the patch may be readhered to the skin after being used, if desired.

In one embodiment, the adhesive patch comprises, consists essentially of, or consists of a water-containing gel. The gel may be coupled to a gas-permeable substrate, such as a fibrous material, for example, a fibrous or fiber material as described elsewhere herein. For example, the adhesive patch may consist essentially of a water-containing gel coupled to a sheet or pad of fibrous material, for example, woven or unwoven fibrous material. The fibrous material can be naturally occurring, synthetic, for example, man made polymeric fibrous materials, combinations of naturally occurring and synthetic fibrous materials and the like.

In one useful embodiment, the water-containing gel is coupled to a cotton fiber sheet. The water-containing gel may comprise one or more water swellable polymeric components swelled with water. Such water swellable polymeric components are well known and are often referred to as hydrophilic polymeric components or materials, or hydrogel-forming polymeric components or materials. One very useful water swellable polymeric component includes one or more polyacrylic acid components and the like. However, it should be noted that one or more other suitable water swellable polymeric components may be used or included in the present cooling devices, and such other water swellable polymeric component or components are included within the scope of the present invention.

The water-containing gel may have a water content sufficient to swell the gel, for example, in a range of about 40% or about 60% or about 75% to about 95% by weight of the gel. One example of a cooling device 12 which comprises a water-containing gel coupled to a gas permeable substrate is disclosed in U.S. Pat. Nos. 6,224,899; 6,228,376, and 6,524,612, and which is publicly available under the tradename Be Koool® (Kobayashi Pharmaceutical Co., LTD. Corporation, Japan). To protect the water-containing gel during shipment and/or from premature activation, a protective, water impermeable layer is placed over the gel layer and may be secured to the fiber sheet. This water impermeable layer is removed from the gel prior to applying the gel to a woman's skin. Advantageously, the gel layer is placed in direct contact with the woman's skin.

The cooling device 12 may be substantially free of a pharmaceutical agent, such as a pharmaceutical agent used in the treatment of menopause symptoms, such as hot flashes. Alternately, the cooling device 12 may include an effective amount of one or more pharmaceutical agents which can be topically administered to the woman while the cooling device is in contact with the woman's skin. Such therapeutic agent or agents may be included in the cooling devices of the present invention using any suitable incorporation technique or techniques effective to provide the therapeutic agent or agents in the cooling device in a form which can be effectively administered to the woman and has no significant adverse effect on the cooling device or on the functioning of the cooling device. For example, and without limitation, such therapeutic agent or agents can be combined with one or more components, for example, and without limitation, with the water, of the cooling device during manufacture.

Such therapeutic agents may be employed to further mitigate against one or more symptoms of hot flashes and/or menopause. In one embodiment, the therapeutic agent employed, if any, is a non-hormonal therapeutic agent. Non-limiting examples of useful therapeutic agents include analgesics, such as aspirin, one or more non-steroidal anti-inflammatory drugs (NSAIDS) and the like; one or more herbal agents, such as chamomile, soy, black cohosh and the like which may be effective, for example, in calming, relaxing and/or otherwise beneficially affecting the physical and/or emotional health and/or well being of the woman; one or more other therapeutic components effective in treating, or at least mitigating against, hot flashes and/or one or more symptoms of hot flashes and/or in calming, relaxing and/or otherwise beneficially affecting the physical and/or emotional health and/or well being of the woman; and the like and mixtures thereof. The therapeutic component employed, if any, advantageously is suitable for transdermal administration from the cooling device through the skin of the woman on whose body the cooling device has been placed.

In one embodiment, cooling devices in accordance with the present invention are provided which have non-rectangular shapes that is which have shapes other than rectangular shapes. Such non-rectangular shapes are particularly useful for placement on certain hot flash origin sites of a woman's body. Because of thee contours and structure of such body areas, non-rectangular shapes are highly advantageous and are more flexible in that they can be used in a greater variety of locations on the woman's body. The cooling devices can be provided in different shapes, for example, in the same sealed package 16, to more effectively accommodate various sites on the woman's body.

As shown in FIGS. 2, 3 and 4, the cooling device 12 is approximately “T-shaped”. The cooling device may have a width, such as the width 22W, of about 3 inches, a height of about 2 inches, and a thickness of about ⅛ of an inch. The width 24W may be about 1 inch.

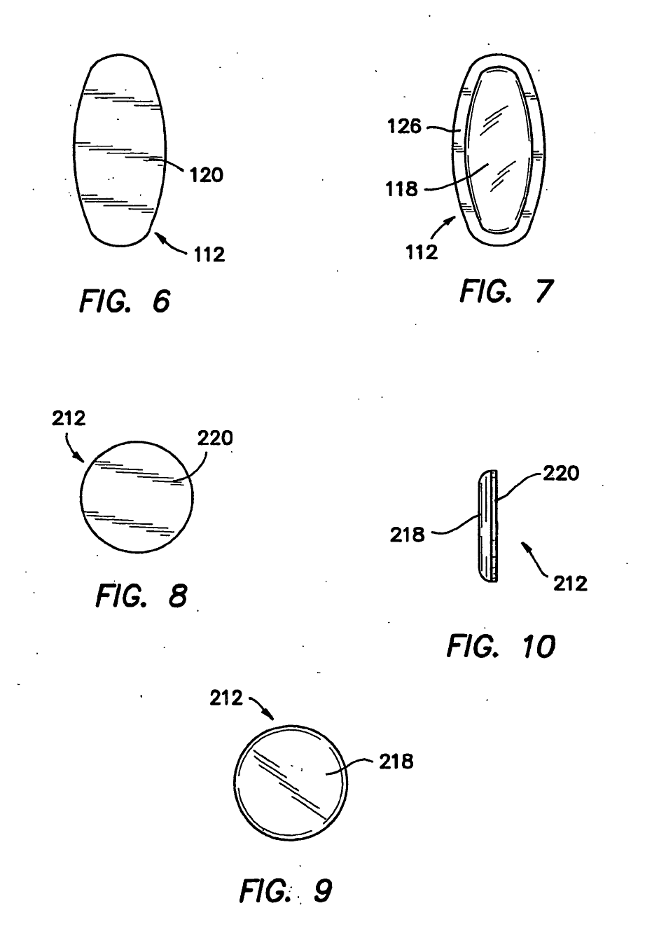

Of course, the cooling device 12 may have any suitable shape, for example, rectangular and non-rectangular shapes, and all such shapes are within the scope of the present invention. For example, as shown in FIGS. 6 and 7, an additional cooling device, shown at 112, has an oval or ovoid shape. The additional cooling device 112 is structured and functions similarly to cooling device 12. The primary difference between the two cooling devices 12 and 112 is the shape. Additional cooling device 112 includes a substrate 120, a cooling component 118 and a border 226 which is free of the cooling component.

Further, as shown in FIGS. 8 to 10, a further cooling device, shown at 212, has a circular shape. Except as expressly described herein, further cooling device 212 is structured and functions similarly to cooling device 12.

One difference between the two cooling devices 12 and 212 is the shape, with cooling device 12 being T-shaped and further cooling device 212 being circularly shaped. In addition, while cooling device 12 includes a peripheral border 26 which is free of the cooling component 18, the cooling component 218 of further cooling device 212 extends to the outer periphery of the substrate 220, as best shown in FIG. 10.

In one embodiment, the cooling device may have a contact surface having any suitable shape, preferably non-rectangular shape useful for providing the desired treatment of one or more symptoms of hot flashes to a woman. Such shapes include, without limitation, rectangular, oval or ovoid, T-shape, circular, polygonal shapes or irregular shapes, other non-rectangular shapes and the like.

The instructions 14 of the kit 10 may include directions to apply the at least one cooling device 12 to the desired region of skin within the first five minutes or less of feeling a hot flash and/or in anticipation of a hot flash occurring. Directions may be in the form of words, pictures, and combinations thereof. The instructions 14 may also include directions to apply the at least one cooling device to a region located between the C3 vertebrae and the T6 vertebrae.

While not wishing to be bound by any particular theory or mechanism of action, it has been discovered that placing the cooling device 12 in an origin site of a hot flash, for example, in a region of the upper back between the shoulder blades, can substantially reduce at least one symptom of hot flashes associated with menopause, and even substantially alleviate hot flashes associated with menopause. In certain situations, it has been found that placement of a cooling device 12 at a region in proximity to the cervical and thoracic vertebrae substantially at the onset of a hot flash or even in anticipation of a hot flash can effectively prevent the hot flash from spreading throughout a woman's body.

As shown in FIG. 5, a cooling device 12′ is placed in an upper region of a woman's back, such as a region between the woman's shoulder blades. Cooling device 12′ is structured and functions similarly to cooling device 12. One difference between cooling device 12′ and cooling device 12 is shape. Specifically, cooling device 12′ has an irregular shape, a generally I-shape, to more easily or readily conform to the region of a woman's back, as shown in FIG. 5.

In accordance with the present invention, a method of treating hot flashes associated with menopause comprises providing at least one cooling device, such as the cooling devices disclosed elsewhere herein; and instructing a woman to place the at least one cooling device on her skin at a location, for example, between her shoulder blades, and advantageously at the hot flash origin site, when a hot flash begins or in anticipation of a hot flash. The cooling device or cooling devices may be placed in one or two or more regions of the woman's body, for example, on the upper portion of the woman's back between and up from the shoulder blades. For example, a cooling device may be placed about four inches up from the shoulder blades and just below the nape of the neck and the spinal column protrusions. As understood by persons of ordinary skill in the art, the precise location may vary depending on the size of the individual, the size of the cooling device, and the origin site of the hot flash. When the cooling device is formed in a “T-shaped” configuration, the cooling device can be placed on the back in an upright configuration or an inverted configuration. When the cooling device is formed in an oval or ovoid shape, the cooling device can be placed with the length of the oval or ovoid shape horizontal, vertical or between horizontal and vertical.

The cooling device can be effectively worn during the day or during the night without causing discomfort to the wearer. During the day, the woman can wear the cooling device shielded by or under her clothes without being noticed by other people. During the night, the woman can wear the cooling device to control night sweats and hot flashes.

The cooling device is preferably secured to the woman's skin for more than one minute to about 2 hours or about 4 hours or about six hours or longer. By placing the cooling device on the skin before, at or during the onset of a hot flash, one or more symptoms of the hot flash can be effectively alleviated and prevented from spreading through the body. However, the cooling device 12 may be applied at any time a woman is experiencing a hot flash. Typically, the cooling device 12 is effective in controlling, that is in reducing, mitigating, alleviating and the like, one or more of the hot flash symptoms within about 1 to about 2 or about 5 or about 10 minutes of contact with the skin.

The present kits and systems may be made by placing the cooling device and the instructions in a package, and sealing the package. If a plurality of cooling devices is to be included in a single outer package, such single outer package can be resealable, to allow the removal and use of one or more cooling devices from the outer package while keeping the remaining cooling devices in the package fresh or substantially undisturbed and ready for future use. Alternately, each individual cooling device can be packaged in its own sealed package and the plurality of sealed packages can be included in an outer, preferably sealed outer, package.

All references, articles, publications and patents and patent applications cited herein are incorporated by reference in their entireties.

While this invention has been described with respect to various specific examples and embodiments, it is to be understood that the invention is not limited thereto and that it can be variously practiced within the scope of the following claims.

1-32. (canceled)

33. A method of treating hot flashes associated with menopause in a woman, comprising:
providing at least one cooling device; and
instructing a woman to place the at least one cooling device at a location on the back of the woman who is experiencing a hot flash or is prone to experiencing hot flashes.

34. (canceled)

35. The method of claim 33, wherein the at least one cooling device comprises an adhesive patch.

36. The method of claim 35, wherein the at least one cooling device comprises a water-containing gel coupled to a gas-permeable substrate.

37. (canceled)

38. The method of claim 36, wherein the water-containing gel comprises a polyacrylic acid component.

39. The method of claim 36, wherein the at least one cooling device comprises one or more strips located at least partially around the water-containing gel, the strips being substantially free of the water-containing gel.

40. (canceled)

41. The method of claim 33, wherein the at least one cooling device is free of a pharmaceutical agent.

42. (cancelled)

43. (canceled)

44. The method of claim 33, comprising instructing the woman to apply the at least one cooling device at a location between the shoulder blades of the woman.

45. The method of claim 33, comprising instructing the woman to apply the at least one cooling device to a region located between the C3 vertebrae and the T6 vertebrae.

46. (canceled)

47. A method of treating hot flashes associated with menopause in a woman, comprising:
placing at least one cooling device at a location on the back of a woman who is experiencing a hot flash or is prone to experiencing hot flashes, the cooling device comprising a water-containing gel.

48. The method of claim 47 further comprising the step of removing the cooling device from a package prior to the step of placing the at least one cooling device at a location on the back.

49. The method of claim 47 wherein the at least one cooling device comprises an adhesive patch.

50. The method of claim 47 wherein the at least one cooling device further comprises a gas-permeable substrate coupled to the water-containing gel.

51. The method of claim 47 wherein the water-containing gel comprises a polyacrylic acid component.

52. The method of claim 47 wherein the at least one cooling device comprises one or more strips located at least partially around the water-containing gel, the strips being substantially free of the water-containing gel.

53. A method of treating hot flashes associated with menopause in a woman, comprising:
removing at least one cooling device from a package; and
placing the at least one cooling device at a location on the back of a woman who is experiencing a hot flash or is prone to experiencing hot flashes.

54. The method of claim 53 wherein the cooling device comprises a water-containing gel.

55. The method of claim 53 wherein the at least one cooling device comprises an adhesive patch.

56. The method of claim 53 wherein the at least one cooling device further comprises a water-containing gel coupled to a gas-permeable substrate.

57. The method of claim 54 wherein the water-containing gel comprises a polyacrylic acid component.

58. The method of claim 54 wherein the at least one cooling device comprises one or more strips located at least partially around the water-containing gel, the strips being substantially free of the water-containing gel.
